(19) United States
(12) Patent Application Publication
(10) Pub. No.: US 2004/0037892 A1
(43) Pub. Date: Feb. 26, 2004

(54) Dietary composition containing conjugated linoleic acid and calcium for improved health

(76) Inventor: Adrianne Bendich, Parsippany, NJ (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/433,038

(22) PCT Filed: Nov. 19, 2001
(86) PCT No.: PCT/US01/44871

(51) Int. Cl. A61K 33/06; A61K 31/714; A61K 31/375; A61K 31/4415; A61K 31/355; A61K 31/202

(52) U.S. Cl. 424/682; 514/52; 514/350; 514/458; 514/474; 514/560

(57) ABSTRACT

The invention provides a composition for oral administration comprising a mixture of conjugated linoleic acid (CLA), docosahexaenoic acid “DHA”, vitamin E, vitamin C, vitamin B6, vitamin B12, folic acid, and calcium together with a suitable carrier. These compositions are particularly useful as dietary supplements administered to reduce the risk factors of cardiovascular disease, such as elevated serum cholesterol levels and high blood pressure.

### FIELD OF THE INVENTION

The present invention relates generally to a dietary supplement, and more particularly, to an oral supplement.

### BACKGROUND OF THE INVENTION

Cardiovascular disease (CVD) is generally recognized to be the primary killer of men and women in developed countries globally. The cost of these premature deaths is great both to the individuals and their families and to the health care system of the country as a whole. The risk factors for cardiovascular disease are well-recognized and include: higher than average serum cholesterol, elevated levels of LDL; a low level of HDL in proportion to the LDL level; higher than average serum triglycerides; higher levels of lipid oxidation products creating plaques and streaks which cause blockages of coronary arteries. Another CVD risk factor, high blood pressure is also a risk factor for strokes.

Research has shown that reduction in these risk factors also reduces the risk of cardiovascular disease and its many costs. [See A. Bendich, R. J. Deckelbaum, eds. Preventive Nutrition: The Comprehensive Guide for Health Professionals. Totowa, N.J.: Humana Press (2000); for example, K. C. Hayes. “Dietary Fat and Coronary Heart Disease.”]

Dietary supplements are well known and recent research has uncovered a number of therapeutic uses therefor. For example, conjugated linoleic acid (CLA), a fatty acid found primarily in milke fat, in has been found to be a naturally occurring anticarcinogen. [See, S. Reiner, “CLA: Does Fat Have a Silver Lining?”, Health Priorities, 8(4): (1996), American Council on Science and Health]. Vitamin E is the major lipid-soluble antioxidant in the human body [L. Mosca, et al., “Antioxidant nutrient supplementation reduces the susceptibility of low density lipoprotein to oxidation in patients with coronary artery disease,” J Am Coll Cardiol, 30:392-9 (1997)]. Vitamin C is another well-known anti-oxidant. See A. Bendich, L. Langseth, “The health effects of vitamin C supplementation: A Review” J. Am. Coll. Nutr. 14:124-36 (1995), [published errata appear in J Am Coll Nutr June:14(3):218 (1995) and August:14(4):398 (1995)]. A variety of benefits have been described in connection with omega-3 fatty acids [W. E. Connor and S. L. Conner, “N-3 Fatty Acids from Fish and Plants: Primary and Secondary Prevention of Cardiovascular Disease”, in: A. Bendich and R. J. Deckelbaum, eds. Preventive Nutrition]. Similarly, benefits of dietary supplementation with folic acid, vitamin B6 and vitamin B12 have been described [S. A. Beresford and C. J. Boushey, “Homocyst(e)ine, Folic Acid and Cardiovascular Disease Risk”, In: in: A. Bendich and R. J. Deckelbaum, eds. Preventive Nutrition]. Calcium status has also been found to be inversely associated with blood pressure. High blood pressure is another important risk factor for cardiovascular disease [D. A. McCarron and M. E. Reusser, “Finding consensus in the dietary calcium-blood pressure debate,” J. Am. Coll Nutr., 18:398S405S (1999)].

There have been dietary compositions described in the past which contain specific vitamins or other supplements, either alone or in a variety of combinations. Many dietary supplements have been described in the art, but their efficacy in preventing cardiovascular disease remains inadequate. As a result, in the field of CVD prevention, there is no single prior art composition which reduces the variety of risk factors associated with this pervasive disease and which has wide spread applicability to the population in developed countries.

### SUMMARY OF THE INVENTION

In one aspect, the present invention provides a novel composition which may be incorporated into an orally administered dietary supplement for the reduction of risk factors associated with CVD. The dietary composition of the invention represents a unique combination of active dietary factors (essential nutrients and non-essential food components) that have never before been developed into a single supplement. This combination is surprisingly effective in the treatment of a variety of risk factors which have been linked to heart attacks, particularly reduction of overall serum cholesterol levels, reductions in high blood pressure, increase in the HDL:LDL ratio, reduction of triglycerides and homocysteine levels, and prevention of lipid oxidation and the formation of plaques and streaks.

In one particular embodiment, the composition of this invention comprises the following dietary components: conjugated linoleic acid (CLA); vitamin E; vitamin C; docasahexanoic acid “DHA”; folic acid; vitamin B6 and vitamin B12, and calcium. In combination, each of these components, which independently reduce one or more of the risk factors for CVD, work synergistically to reduce the risk of CVD more effectively than any of these components taken alone. Additionally, all of the components have wide safety margins, therefore it is expected that the combination of all of these active components will require a lower concentration of each component alone, and therefore, enhance the safety of the combination of these dietary factors.

In another aspect, the invention provides a pharmaceutical and/or dietary composition containing the formulation described above in admixture with pharmaceutically acceptable base, and optionally containing other known agents including, but not limited to stabilizer agents, preservatives and emulsifiers. The compositions, according to this invention may be presented in different embodiments, including but not limited to tablets, powders, chews, bars, and shakes or similar formulations.

In a further aspect of this invention, a method is provided for preparing the novel dietary compositions described herein and incorporating the same into orally administrated pharmaceutical compositions.

In yet a further aspect, this invention provides a process for treating individuals to reduce the risk factors for CVD comprising orally administering a pharmaceutical composition as described above.

Other aspects and advantages of the present invention will become apparent from the following detailed description thereof.

### DETAILED DESCRIPTION OF THE INVENTION

The present invention provides novel compositions comprised of combinations of selected mixtures of active dietary factors, including certain vitamins and other components, which are surprisingly effective in their ability to reduce the risk factors of CVD and promote improved cardiovascular health. The oral administration of these compositions acts to reduce serum cholesterol levels and blood pressure, increase HDL levels in proportion to LDL levels, to protect lipids from oxidation thereby preventing the formation of plaques and streaks which block coronary arteries, and to lower both triglyceride levels and homocysteine levels. In addition, it is believed that oral administration of the compositions of this invention acts to reduce the risk of stroke, as well as heart attack, in human adults.

The orally-administered compositions of this invention include those dietary admixtures in which the formulations is swallowed in any acceptable form. Conventional forms for this purpose include but are not limited to liquids, tablets, effervescent tablets, pills, powders, wafers or premixed shakes. See Remington's Practice of Pharmacy, 11^{th} Edition, (1956).

The novel compositions of the present invention are comprised of the following vitamins and dietary factors which in combination provide a surprising result in reducing the risk factors of CVD. The combination of dietary factors and vitamins work synergistically to improve cardiovascular health to a great degree than expected. The essential components of the compositions are CLA, docosahexaenoic acid “DHA”, vitamin E, vitamin C, folic acid, vitamin B6, vitamin B12, and calcium.

Suitably, the conjugated linoleic acid useful in the present invention refers to any member of a group of several variants of linoleic acid (also termed octadecadienoic acid). CLA may be isolated from foods such as cheese and milk using methods known to those of skill in the art. Alternatively, CLA may be synthesized using known techniques. For example, CLA may be synthesized from 95% pure linoleic acid [D. DeVoney et al., “Trans-10,Cis-12 Octadecadienoic acid increases lymphocyte proliferation”, p. 56, 1998 Annual Report of the Food Research Institute]. Such synthesized CLA typically contains 43% c9, t11/t9,c11 and 44% t10,c12 octadecadienoate. CLA may also be purchased from a variety of commercial sources, e.g., CLA enriched for c9,t11/t9,c11 (70.5% with no t10,c12 is available form Matreya, Inc); CLA may also be purchased from Peak Nutrition, Syracuse, Nebr. Typically, CLA is present in the composition of the invention in an amount of about 250 mg to about 3000 mg; more desirably, about 300 mg to about 2000 mg, and most preferably, about 500 mg to about 1000 mg per dose. Advantageously, CLA is contributes to the ability of the composition of the invention to protect against cancer, and to modulate the immune system.

The composition of the invention further includes vitamin E, which may be in any suitable form. Natural vitamin E can be isolated from vegetable oils, including corn, cottonseed, rapeseed, peanut, sunflower and soybean oil, or obtained from a variety of commercial sources. Natural vitamin E may be in the form dalpha-tocopherol (RRR-alpha-tocopherol), or the acetate [d-alpha-tocopheryl acetate (RRR-alpha-tocopheryl acetate)] or succinate salt thereof [d-alpha-tocopheryl acid succinate (RRR-alpha-tocopheryl acid succinate)], or may be in the form of natural mixed tocopherols [d-alpha-, d-beta-, d-gamma and d-delta-tocopherol]. Alternatively, synthetic vitamin E may be produced from petrochemicals in the form of dl-alpha-tocopherol (all-rac-alpha-tocopherol), or the acetate [dl-alphatocopheryl acetate, (all-rac-alpha-tocopheryl acetate)] or succinate salt thereof [dlalpha-tocopheryl acid succinate (all-rac-alpha-tocopheryl acid succinate), or mixtures thereof. Where reference is made herein to vitamin E, any of the natural or synthetic forms of the vitamin may be used, or combinations thereof. In the formulation of the invention, Vitamin E prevents the blockage of coronary arteries and other vessels within the body that result when oxidized lipids are permitted to form [S. B. Kritchevsky et al, “Dietary antioxidants and carotid artery wall thickness”, The ARIC Study. Atherosclerosis Risk in Communities Study, Circulation, 92:2142-50 (1995)]. Thus, vitamin E is a useful as an antioxidant [Jeng et al, Am. J. Clin. Nutr., 64:960-5 (1996); F. M. Steinberg and A. Chait, Am. J. Clin. Nutr., 68:319-27 (1998), [publ. erratum appears in Am J Clin Nutr., June:69(6):1293 (1999)] Thus, vitamin E clearly is an important component in the formulation of the composition designed for the prevention of CVD. In one embodiment, the composition of the invention contains about 50 IU to about 800 IU of vitamin E, and most preferably, about 100 IU.

Vitamin C is another component of the composition of the invention. Vitamin C is an anti-oxidant which works synergistically with Vitamin E to protect cellular components from oxidative damage leading to cardiovascular disease. Vitamin C optimizes the effects of Vitamin E to reduce the oxidation of lipids. Further, Vitamin C taken alone has been linked with a decreased risk of CVD as well as CVD mortality, possibly because of the reduction in systolic and diastolic blood pressure seen in those individuals taking large does of the vitamin [S. J. Duffy et al, “Treatment of hypertension with ascorbic acid”, Lancet, 354:2048-9 (1999); P. Weber et al, “Vitamin C and human health—a review of recent data relevant to human requirements”, Int. J. Vitam. Nutr. Res., 66:19-30 (1996)]. In one suitable embodiment, vitamin C is in the form of ascorbate or ascorbic acid, and is present in an amount of about 60 mg to about 1000 mg, or about 100 to about 500 mg.

The present invention further includes an omega-3 fatty acid which is known to cause reduction in triglycerides and increase in HDL levels. Most preferably, this omega-3 fatty acid is in the form of docasahexaenoic acid “DHA”, which may be extracted from algae using known methods or purchased commercially. DHA is the longest omega-3 fatty acid and is known to be important in the functioning of every cell membrane in the body. It is found in especially high concentration in the human brain and retina. DHA has also been seen to reduce the risk of ventricular arrythmia that can result in sudden death. In one desirable embodiment, the composition of the invention contains about 125 mg to about 500 mg, and preferably about 230 to about 250 mg DHA. Alternatively, another omega-3 oil may be included in the composition of the invention.

Folic acid (or a pharmaceutically acceptable salt thereof), vitamin B6 and vitamin B12 are all involved in normal amino acid metabolism. Specifically, these vitamins have been known to significantly reduce elevated homocysteine levels that have been linked to an increased risk of CVD, as well as stroke, peripheral vascular disease and dementia. Most suitably, the composition of the invention contains about 400 μg to about 1000 μg folic acid (or folate); about 2 mg to about 50 mg vitamin B6, preferably about 10 to 25 mg vitamin B6; and about 6 μg to about 1 mg vitamin B12.

Calcium has been associated with reducing systolic and diastolic blood pressure. The composition of this invention contains about 200 to about 100 mg of elemental calcium, which may be in the form of pharmaceutically acceptable salt thereof. In one desirable embodiment, calcium is present in the composition of the form of calcium carbonate.

Optionally, the eight active components discussed above may be admixed with other active ingredients. Preferably, however, these components are the only active ingredients in the composition of the invention.

One particularly desirable embodiment of the composition of the invention is provided in Example 1 below. However, the invention is not limited by this formulation, or by the ranges provided herein, which are intended for guidance only. One of skill in the art can readily select other ranges, depending upon the delivery form (e.g., effervescent tablet vs. tablet), the age, and condition of the patient, among other factors.

A composition of the formulation of the invention may be used orally to treat and/or prevent risk factors of CVD and stroke, including reduction of high blood pressure and overall serum cholesterol.

While not wishing to be bound by theory, the inventors believe that the compositions work by acting at different sites and aspects of cardiovascular disease. High cholesterol, high LDL, elevated triglycerides, high blood pressure, low HDL, high homocysteine levels and oxidized lipids are all attacked by one or more of the dietary factors present in the oral formulation and act synergistically to reduce the risk factors of CVD. By affecting CVD risk factors at several sites and by different mechanisms of action, there is an enhancement of the effects of the supplement that is greater than the additive effect of the dietary factors. The dietary supplement contains active ingredients that are safe, efficacious and cost-effective in lowering CVD risk factors.

The compositions of the present invention are preferably presented for administration to humans and animals in unit dosage forms, such as tablets, capsules, pills, powders, granules, and oral solutions or suspensions and the like, containing suitable quantities of an active ingredient. For oral administration either solid or fluid unit dosage forms can be prepared.

Powders are prepared quite simply by comminuting the active ingredient(s) to a suitably fine size and mixing with a similarly comminuted diluent. The diluent can be an edible carbohydrate material such as lactose or starch. Advantageously, a sweetening agent or sugar is present as well as a flavoring oil.

Capsules are produced by preparing a powder mixture as hereinbefore described and filling into formed gelatin sheaths. Advantageously, as an adjuvant to the filling operation, a lubricant such as a talc, magnesium stearate, and the like is added to the powder mixture before the filling operation.

Soft gelatin capsules are prepared by machine encapsulation of a slurry of active ingredients with an acceptable vegetable oil, light liquid petrolatum or other inert oil or triglyceride.

Tablets, chews and bars are made by preparing a powder mixture, granulating or slugging, adding a lubricant and pressing into tablets, chews, or bars. The powder mixture is prepared by mixing an active ingredient, suitably comminuted, with a diluent or base such as starch, lactose, kaolin, dicalcium phosphate and the like. The powder mixture can be granulated by wetting with a binder such as corn syrup, gelatin solution, methylcellulose solution or acacia mucilage and forcing through a screen. As an alternative to granulating, the powder mixture can be slugged, e.g., run through the tablet, bar or chew, machine and the resulting imperfectly formed tablets broken into pieces (slugs). The slugs can be lubricated to prevent sticking to the shape-forming dies by means of the addition of stearic acid, a stearic salt, talc or mineral oil. The lubricated mixture is then compressed into tablets, chews or bars, as desired. Optionally, a tablet can be provided with a protective coating consisting of a sealing coat or enteric coat of shellac, a coating of sugar and methylcellulose and polish coating of carnauba wax. Advantageously, chews and bars may be mixing with a variety of flavorings, sweetening agents, or the like.

Fluid unit dosage forms for oral administration such as syrups, elixirs and suspensions can be prepared wherein each teaspoonful of composition contains a predetermined amount of active ingredient for administration. The water-soluble forms can be dissolved in an aqueous vehicle together with sugar, flavoring agents and preservatives to form a syrup. An elixir is prepared by using a hydroalcoholic vehicle with suitable sweeteners together with a flavoring agent. Suspensions can be prepared on the insoluble forms with a suitable vehicle with the aid of a suspending agent such as acacia, tragacanth, methylcellulose and the like.

In another embodiment, the invention provides a method of using the composition to improve the health of the heart and to reduce risk factors associated with cardiovascular disease by delivering to an individual the composition of the invention. Thus, delivery of the composition of the invention, e.g., by oral administration, is useful for preventing oxidation of low density lipoprotein (LDL), increasing high density lipoprotein (HDL), and for reducing total cholesterol. Delivery of the composition of the invention is also useful for reducing triglycerides and reducing homocysteine.

Desirably, the compositions of the invention are formulated such that an effective amount is delivered by two tablets (or other suitable formulation) a day. Suitably, these doses may be taken with meals, mixed into feed, or taken on an empty stomach. Generally improvement is observed after two weeks of daily use.

Several factors have been observed to interfere with the positive effects of dietary supplementation with the compositions of the invention, including smoking, eating a high fat diet, omitting dietary fibers or roughage from a daily diet and maintaining an essentially sedentary lifestyle.

The compositions of the present invention, in addition to their use in treating CVD in humans, may also be useful in treating non-human animals, particularly mammals. For example, these dietary supplements may be useful for companion animals such as dogs and cats, for cattle, horses, and pigs, among other animals.

The following example which demonstrates the compositions of the invention for illustrative purposes only and does not limit the scope of the invention. The compositions of this invention are anticipated to produce surprisingly good results in reducing a variety of risk factors associated with impaired cardiovascular conditions. As demonstrated in the following example, the compositions of the invention have advantages over the prior art in safely lowering CVD risk factors in a cost effective manner.

### EXAMPLE

In one exemplary embodiment, the components listed below were combined into a tablet, using simple mixing procedures.

TABLE 1

| Component | Amount | |
|---|---|---|
| CLA | 500 | mg |
| DHA | 125 | mg |
| Vitamin E | 50 | IU |
| Vitamin C | 60 | mg |
| Folic Acid | 400 | μg |
| Vitamin B6 | 20 | mg |
| Vitamin B12 | 6 | μg |
| Calcium | 100 | mg |

The above ingredients all or in part can be:

1. Mixed dried (direct compression process-DCP) with well recognized tableting aid(s)/filler(s), binding agent(s), disintegrant (s) and lubricant(s), as necessary or desired to form a blend that can be directly compressed into tablets; or

2. Wet granulated (Wet Granulation Process-WGP) with well recognized tableting aid(s)/filler(s), granulating agent(s), disintegrant(s) and lubricant(s) as necessary or desired to form a blend that it can be directly compressed into tablets.

Numerous modifications of this invention are encompassed by the above description and the scope of the following claims. For example, other suitable optional ingredients may be employed in the composition of this invention which are obvious to one of skill in the art considering the present disclosure. Similarly other systemic disorders other than those described may be treated with the compositions of this invention. It should be understood therefore that various changes may be made in the products and processes herein described without significantly affecting the resultant formulations or their use in medical treatment. Various modifications in conditions of preparation such as time and temperature, or changes in administrative procedure or dosages differing from those given herein as illustrative of the preferred embodiments of the invention, may be made without departure from the scope of the invention envisioned by the inventor.

All publications, including but not limited to patents and patent applications, cited in this specification are herein incorporated by reference as if each individual publication were specifically and individually indicated to be incorporated by reference herein as though fully set forth.

What is claimed is:

1. A composition for oral administration comprising conjugated linoleic acid (CLA), an omega-3 fatty acid, vitamin E, vitamin C, vitamin B6, vitamin B12, folic acid and calcium.

2. The composition according to claim 1 wherein the composition is a stable emulsion.

3. The composition according to claim 1, wherein said mixture contains about 300 mg to about 2000 mg CLA.

4. The composition according to claim 3, wherein said mixture contains about 500 mg CLA.

5. The composition according to claim 1, wherein the omega-3 fatty acid is docosahexaenoic acid (DHA) which is present in an amount of about 125 mg to about 500 mg DHA.

6. The composition according to claim 5, wherein said mixture contains about 125 mg DHA.

7. The composition according to claim 1, wherein said mixture contains about 50 IU to about 500 IU vitamin E.

8. The composition according to claim 7, wherein said mixture contains about 100 IU vitamin E.

9. The composition according to claim 1, wherein said mixture contains about 60 mg to about 1000 mg vitamin C.

10. The composition according to claim 9, wherein said mixture contains about 60 mg vitamin C.

11. The composition according to claim 1, wherein said mixture contains about 400 μg to about 1000 μg folic acid.

12. The composition according to claim 11, wherein said mixture contains about 400 μg folic acid.

13. The composition according to claim 1, wherein said mixture contains about 2 mg to about 50 mg vitamin B6.

14. The composition according to claim 13, wherein said mixture contains about 20 mg vitamin B6.

15. The composition according to claim 1, wherein said mixture contains about 6 μg to about 1 mg vitamin B12.

16. The composition according to claim 1, wherein said mixture contains about 6 μg vitamin B12.

17. The composition according to claim 1, wherein said mixture contains about 200 mg to 1000 mg calcium.

18. The composition according to claim 1 wherein the mixture is in a solid dosage form which is a tablet, capsule, soft gelatin capsule, or sachet.

19. A method for reducing risk factors associated with cardiovascular disease in a mammal in need thereof, which method comprised orally administering to said mammal a composition according to any one of claims 1 to 17.

20. A method for preventing oxidation of low density lipoprotein in a mammal in need thereof, which method comprised orally administering to said mammal a composition according to any one of claims 1 to 17.

21. A method for increasing high density lipoprotein in a mammal in need thereof, which method comprised orally administering to said mammal a composition according to any one of claims 1 to 17.

22. A method for reducing total cholesterol in a mammal in need thereof, which method comprised orally administering to said mammal a composition according to any one of claims 1 to 17.

23. A method for reducing triglycerides in a mammal in need thereof, which method comprised orally administering to said mammal a composition according to any one of claims 1 to 17.

24. A method for reducing homocysteine in a mammal in need thereof, which method comprised orally administering to said mammal a composition according to any one of claims 1 to 17.
